SEQUENCE LISTING (5 sequences)

SEQ ID NO: 1 - protein, 386 aa, Caloenas nicobarica
MGSIGAASTEFCFDAFNELKVQYVDQNIFFAPLSILSALSIIYLGARENTKAQIDKVVHFDKITGSGETIESQCGTSINVHTSLKDMFIQITKPSDNYSASFASRLYAEETYPILPEYLQCVKELYKEGLETVSFQTAADQARELINSWVESQTNGMIRNILQPGSVGPQTEMVLVNAIYFKGVWEKAFKDEDTQTVPFRLTEQESKPVEMMYQAGSFRVAVLASERMKILELPYASGMLSMLVLLPDDVSGLEQLENAITFEKLLEWTSSNLMEERTTKVYLPRMKIEEKYNLTSLLMALGITDLFSSSANLSGISSAESLKVSEAVHEAFVEIYEAGSEVAGSAGAGAEVESASEEFRADHPFLFLIRHNPTNGILFFGRYFSP

SEQ ID NO: 2 - protein, 386 aa, Scopus umbretta
MGSIGAASSEFCFDVFKELKVQHVNENIFYSPLSIISALSMVYLGARENTRAQIDKVVHFDQITGFGETIESQCGTSVSVHTSLKDMFTQITKPSDNYSLSFASRLYAEETYPILPEYIQCVKELYKGGLETISFQTAAEQARELINSWVENQTSGMIKNILQPGSVDSQTEMVLVNAIYFKGIWEKAFKDEDTQAVPFRITEQESKTVQMMYQIGSFNVAVIAAEKIKILELPYTSGELSMLVLLPDDVSGLEQLETAITFEKLVEWTSSNTMEERKIKVYLPRMKIEEKYNLTSVLIALGITDLFSSSANLSGISSAESLKMSEAIHEASVEIYEAGSEVVSSTEAGMEVTSTSEEFRADHPFLFLIKHNPTNSILFFGRCFSP

SEQ ID NO: 3 - protein, 370 aa, Ficedula albicollis
MGSISAANAEFCFDVFKEVKFYRSNDNVLFSSLSMLSTLALVYMGARGKTQSQMEKVLHFDNVTGDGEISDSQCGTPEYIHKSFKDLLSDISRQNATYSLRIGDRLYIEKTYPILEEYIKCAKKFYRAELEEVDFKTAAEEARQLINSWVEKETNGRIQDFLVSDSVDLNTALVFVNVIYFKGIWKTAFKEEHTQEEPFNVTEQESRPVQMMRQNNTLKVARVAEDKIKILELPYASGELSLLVLLPDDISGLAQLENKISYEKLLEWTSPKVMEKKRVRVYLPRMKIEQKYNLTSVLTSLGMTDLFSPRANLSGISSAEGLRISEAIHEAYMEVTEEGTEAGGSEVVTGDIQPLWHLLSREPEGVWSIP

SEQ ID NO: 4 - protein, 24 aa, Synthetic construct
VCVLFHYYIVIVLLLFFLANTGFT

SEQ ID NO: 5 - protein, 26 aa, Synthetic construct
CCVCTVFHHYIVIVLLLFALDNTGFI

(19) United States
(12) Patent Application Publication
(10) Pub. No.: US 2026/0060277 A1
(43) Pub. Date: Mar. 5, 2026

(54) RECOMBINANT PROTEINS, COMPOSITIONS, AND METHODS OF USING SAME

(71) Applicants: EGGMENTED REALITY LTD., Kiryat Shmona (IL); MIGAL GALILEE RESEARCH INSTITUTE LTD., Kiryat Shmona (IL)

(72) Inventors: Itai BLOCH, Rehovot (IL); Itamar YADID, Kibbutz Ayelet HaShachar (IL); Tamar MEIRON, Holon (IL)

(73) Assignees: EGGMENTED REALITY LTD., Kiryat Shmona (IL); MIGAL GALILEE RESEARCH INSTITUTE LTD., Kiryat Shmona (IL)

(21) Appl. No.: 19/105,816

(22) PCT Filed: Aug. 22, 2023
(86) PCT No.: PCT/IL2023/050891

§ 371 Date: Feb. 23, 2025

Related U.S. Application Data
(60) Provisional application No. 63/399,794, filed on Aug. 22, 2022.

(51) Int. Cl. A23J 1/08; A23J 3/20; A23L 15/00; C12P 21/02

(52) U.S. Cl. CPC A23J 1/08; A23J 3/20; A23L 15/35; C12P 21/02

(57) ABSTRACT

The present invention provides an edible composition including at least one: recombinant pigeon ovalbumin, recombinant hamerkop ovalbumin, or recombinant collard flycatcher ovalbumin. Further provided are a food product including the edible composition of the invention, as well as a method for preparing same.

### CROSS-REFERENCE TO RELATED APPLICATIONS

This application claims the benefit of priority of U.S. Provisional Patent Application No. 63/399,794, titled “RECOMBINANT PROTEINS, COMPOSITIONS, AND METHODS OF USING SAME”, filed on Aug. 22, 2022, the contents of which are incorporated herein by reference in their entirety.

### REFERENCE TO AN ELECTRONIC SEQUENCE LISTING

The contents of the electronic sequence listing (EGMT-MIG-P-001-PCT.xml; size: 6,503 bytes; and date of creation: Aug. 18, 2023) is herein incorporated by reference in its entirety.

### FIELD OF INVENTION

The present disclosure relates to, inter alia, recombinant animal-free food compositions comprising recombinant avian ovalbumin, and methods of using same, such as for making a food or an edible composition.

### BACKGROUND

Proteins are important dietary nutrients and food ingredients. They can serve as a fuel source or as sources of amino acids, including the essential amino acids that cannot be synthesized by the body. The daily recommended intake of protein for healthy adults is 10% to 35% of a person's total calorie needs, and currently the majority of protein intake for most humans is from animal-based sources. In addition, proteins are used in a wide variety of foods and food ingredients. In many cases, these proteins are sourced from animals. With the world population growth and the coinciding growth in global food demand, there is a need to provide alternative sustainable, non-animal-based sources of proteins as useful source of protein for daily diet, food ingredients and food products.

### SUMMARY

According to one aspect, there is provided an edible composition comprising at least one of: recombinant pigeon ovalbumin, recombinant hamerkop ovalbumin, collard flycatcher ovalbumin, or any combination thereof.

According to another aspect, there is provided a food product or an ingredient thereof comprising the edible composition disclosed herein.

According to another aspect, there is provided a method for preparing a food product that requires an amount of egg-white protein, the method comprising substituting an amount of egg-white protein required for preparing the food product with an equivalent amount of the edible composition disclosed herein.

In some embodiments, the recombinant pigeon ovalbumin comprises the amino acid sequence set forth in SEQ ID NO: 1, or a functional analog thereof having at least 85% homology thereto.

In some embodiments, the recombinant hamerkop ovalbumin comprises the amino acid sequence set forth in SEQ ID NO: 2, or a functional analog thereof having at least 85% homology thereto.

In some embodiments, the recombinant collard flycatcher ovalbumin comprises the amino acid sequence set forth in SEQ ID NO: 3, or a functional analog thereof having at least 85% homology thereto.

In some embodiments, any one of: the pigeon ovalbumin, recombinant hamerkop ovalbumin, and recombinant collard flycatcher ovalbumin is characterized by having a gelation activity equal to or greater than a gelation activity of a control ovalbumin.

In some embodiments, the edible composition is an egg-white substituent.

In some embodiments, the edible composition is in the form of a dried powder.

In some embodiments, any one of: the pigeon ovalbumin, recombinant hamerkop ovalbumin, and recombinant collard flycatcher ovalbumin is encoded from a polynucleotide being codon optimized for expression in a cell.

In some embodiments, the cell comprises a yeast cell.

In some embodiments, any one of: the pigeon ovalbumin, recombinant hamerkop ovalbumin, and recombinant collard flycatcher ovalbumin is produced or heterologouslly expressed in a yeast cell.

In some embodiments, the yeast cell comprises a Pichia pastoris cell.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a graphs showing that NP-Ovalbumin performs better Gelation then Chicken Ovalbumen/Ovalbumin. Gelation of 400 μl protein solution after heating at 95° C. for 8.5 minutes. Black solid line represents the initial Gelation point. NP—Nicobar pigeon; EW—egg white albumen.

FIG. 2 includes photographs showing that NP-Ovalbumin performs irreversible gelation of a semi-transparent gel. Gelation of 400 μl protein solution after heating at 95° C. for 8.5 minutes.

FIG. 3 includes photographs of protein gel separation showing that fermentation of NP-Ovalbumin produces higher yields compared to FA-ovalbumin and SU-ovalbumin. Upper panel—Coomassie Blue staining; lower panel—western Blot (anti Ovalbumin antibodies).

FIG. 4 includes photographs and graphs showing Gelation differences between recombinant NP, FA and SU-Ovalbumins and commercially available Chicken Ovalbumen/Ovalbumin.

FIG. 5 includes a graph and photographs showing that NP-Ovalbumin has an increase gelation ability compared to SU- and FA-Ovalbumin. Gelation of 400 μl protein solution after heating at 95° C. for 8.5 minutes.

FIG. 6 includes photographs showing that NP-Ovalbumin increased water solubility compared to 98% pure Chicken Ovalbumin in 100 mg/ml solution.

FIGS. 7A-7B include photographs showing that NP-Ovalbumin has a lower foaming ability compared to egg white albumen in 50 mg/ml solutions. (7A) Samples were vortexed for 30 sec (Left) and 60 sec (Right); (7B) Samples were manually mixed.

### DETAILED DESCRIPTION

According to some embodiments, there is provided an edible composition comprising pigeon ovalbumin, recombinant hamerkop ovalbumin, recombinant collard flycatcher ovalbumin, or any combination thereof.

In some embodiments, the edible composition comprises pigeon ovalbumin and recombinant hamerkop.

In some embodiments, the edible composition comprises pigeon ovalbumin and recombinant collard flycatcher ovalbumin.

In some embodiments, the edible composition comprises recombinant hamerkop ovalbumin and recombinant collard flycatcher ovalbumin.

In some embodiments, the edible composition comprises pigeon ovalbumin, recombinant hamerkop ovalbumin, and recombinant collard flycatcher ovalbumin.

In some embodiments, a recombinant pigeon ovalbumin comprises the amino acid sequence: MGSIGAASTEFCFDAFNELKVQYVDQNIFFAPLSILSALSIIYLGARENTKAQIDKV VHFDKITGSGETIESQCGTSINVHTSLKDMFIQITKPSDNYSASFASRLYAEETYPIL PEYLQCVKELYKEGLETVSFQTAADQARELINSWVESQTNGMIRNILQPGSVGPQ TEMVLVNAIYFKGVWEKAFKDEDTQTVPFRLTEQESKPVEMMYQAGSFRVAVL ASERMKILELPYASGMLSMLVLLPDDVSGLEQLENAITFEKLLEWTSSNLMEERT TKVYLPRMKIEEKYNLTSLLMALGITDLFSSSANLSGISSAESLKVSEAVHEAFVEI YEAGSEVAGSAGAGAEVESASEEFRADHPFLFLIRHNPTNGILFFGRYFSP (SEQ ID NO: 1), or a functional analog thereof, having at least 50%, 60%, 70%, 80%, 90%, 95%, 97%, or 99% homology thereof, or any value and range therebetween. Each possibility represents a separate embodiment of the invention.

In some embodiments, a recombinant protein as disclosed herein further comprises a signal peptide. In some embodiments, the recombinant pigeon ovalbumin further comprises a signal peptide comprising the amino acid sequence: VCVLFHYYIVIVLLLFFLANTGFT (SEQ ID NO: 4).

In some embodiments, a recombinant hamerkop ovalbumin comprises the amino acid sequence: MGSIGAASSEFCFDVFKELKVQHVNENIFYSPLSIISALSMVYLGARENTRAQIDK VVHFDQITGFGETIESQCGTSVSVHTSLKDMFTQITKPSDNYSLSFASRLYAEETY PILPEYIQCVKELYKGGLETISFQTAAEQARELINSWVENQTSGMIKNILQPGSVD SQTEMVLVNAIYFKGIWEKAFKDEDTQAVPFRITEQESKTVQMMYQIGSFNVAVI AAEKIKILELPYTSGELSMLVLLPDDVSGLEQLETAITFEKLVEWTSSNTMEERKI KVYLPRMKIEEKYNLTSVLIALGITDLFSSSANLSGISSAESLKMSEAIHEASVEIY EAGSEVVSSTEAGMEVTSTSEEFRADHPFLFLIKHNPTNSILFFGRCFSP (SEQ ID NO: 2), or a functional analog thereof, having at least 50%, 60%, 70%, 80%, 90%, 95%, 97%, or 99% homology thereof, or any value and range therebetween. Each possibility represents a separate embodiment of the invention.

In some embodiments, the recombinant hamerkop ovalbumin further comprises a signal peptide comprising the amino acid sequence: CCVCTVFHHYIVIVLLLFALDNTGFI (SEQ ID NO: 5).

In some embodiments, a recombinant collared flycatcher ovalbumin comprises the amino acid sequence: MGSISAANAEFCFDVFKEVKFYRSNDNVLFSSLSMLSTLALVYMGARGKTQSQM EKVLHFDNVTGDGEISDSQCGTPEYIHKSFKDLLSDISRQNATYSLRIGDRLYIEK TYPILEEYIKCAKKFYRAELEEVDFKTAAEEARQLINSWVEKETNGRIQDFLVSDS VDLNTALVFVNVIYFKGIWKTAFKEEHTQEEPFNVTEQESRPVQMMRQNNTLKV ARVAEDKIKILELPYASGELSLLVLLPDDISGLAQLENKISYEKLLEWTSPKVMEK KRVRVYLPRMKIEQKYNLTSVLTSLGMTDLFSPRANLSGISSAEGLRISEAIHEAY MEVTEEGTEAGGSEVVTGDIQPLWHLLSREPEGVWSIP (SEQ ID NO: 3), or a functional analog thereof, having at least 50%, 60%, 70%, 80%, 90%, 95%, 97%, or 99% homology thereof, or any value and range therebetween. Each possibility represents a separate embodiment of the invention.

In some embodiments, any one of the pigeon ovalbumin and recombinant hamerkop ovalbumin is characterized by having a gelation activity equal to or greater than a gelation activity of a control ovalbumin.

The term “gelation” as used herein is interchangeable with coagulation.

In some embodiments, gelation activity provides utility in applications such as cooked egg compositions.

In some embodiments, gelation activity is induced or obtained by heating.

In some embodiments, heating comprises subjecting to a temperature of 30 to 100° C. In some embodiments, heating comprises subjecting to a temperature of 95° C., as exemplified herein.

In some embodiments, gelation activity is determined over a period of 1 day to 14 days.

In some embodiments, a control ovalbumin comprises a chicken ovalbumin. In some embodiments, a control ovalbumin is a purified ovalbumin. In some embodiments, a control ovalbumin is a synthetic ovalbumin. In some embodiments, a control ovalbumin comprises chicken whole egg white(s) protein. In some embodiments, a control ovalbumin comprises egg white, egg protein, ovalbumin, or whole egg, produced by an animal or collected from an animal, in particular an egg-laying animal such as a bird.

In some embodiments, the edible composition is an egg-white substituent.

In some embodiments, the edible composition is free of animal protein. In some embodiments, the edible composition is free of animal protein.

In some embodiments, the edible composition is in the form of a powder. In some embodiments, the edible composition is in the form of a dry powder. In some embodiments, the edible composition is a concentrate. In some embodiments, the edible composition is a concentrate. In some embodiments, the edible composition is a liquid concentrate.

In some embodiments, any one of the pigeon ovalbumin and recombinant hamerkop ovalbumin as disclosed herein is encoded from a polynucleotide being codon optimized for expression in a cell.

In some embodiments, cell comprises a yeast cell.

In some embodiments, any one of the pigeon ovalbumin and recombinant hamerkop ovalbumin is produced or heterologouslly expressed in a yeast cell. In some embodiments, the recombinant avian ovalbumin is produced and secreted from a yeast cell.

In some embodiments, a yeast cell comprises a Pichia cell. In some embodiments, a yeast cell comprises a Pichia pastoris cell (also known as Komagataella phaffii).

According to some embodiments, there is provided a food product or an ingredient thereof comprising the edible composition disclosed herein.

Non-limiting examples of food products include, but are not limited to, meat product or meat-like product, or fish-like or shell-fish-like products. Exemplary meat and meat-like products include, but are not limited to, burger, patty, sausage, hot dog, sliced deli meat, jerky, bacon, nugget, and ground meat-like mixtures. Meat-like products can resemble beef, pork, chicken, lamb, and other edible and consumed meats for humans and for other animals. Fish-like and shell-fish-like products can resemble, for example, fish cakes, crab cakes, shrimp, shrimp balls, fish sticks, seafood meat, crab meat, fish fillets and clam strips.

According to some embodiments, there is provided a method for preparing a food product that requires an amount of egg-white protein.

In some embodiments, the method comprises substituting an amount of egg-white protein required for preparing the food product with an equivalent amount of the edible composition disclosed herein.

As used herein, the term “recombinant protein” refers to a protein which is coded for by a recombinant DNA and is thus not naturally occurring. The term “recombinant DNA” refers to DNA molecules formed by laboratory methods of genetic recombination. Generally, this recombinant DNA is in the form of a vector, plasmid or virus used to express the recombinant protein in a cell.

In some embodiments, a pigeon is or comprises Caloenas nicobaria (Nicobar pigeon).

In some embodiments, a hamerkop is or comprises Scopus umbrette.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

Out of 800 ovalbumin homologues from different avian species, 7 candidates were transformed and expressed in the food grade yeast Komagataella phaffii (Pichia Pastoris). The 3 candidates described herein are: Scopus umbrette (SU; Hamerkop), Ficedula albicollis (FA; Collared flycatcher) and Caloenas nicobarica (NP; Nicobar pigeon)—Ovalbumin homologues. This expression system is based on transformation of pD912 plasmid obtaining a specific secretion factor (1 out of 10 different S.F tested for each ovalbumin sequence) enabling the secretion of the recombinant protein out of the yeast cell, its accumulation and collection from the fermentation media.

### Example 1

### NP-Ovalbumin has a Greater Coagulation and Gelation Capabilities Compared to Chicken Egg White Products

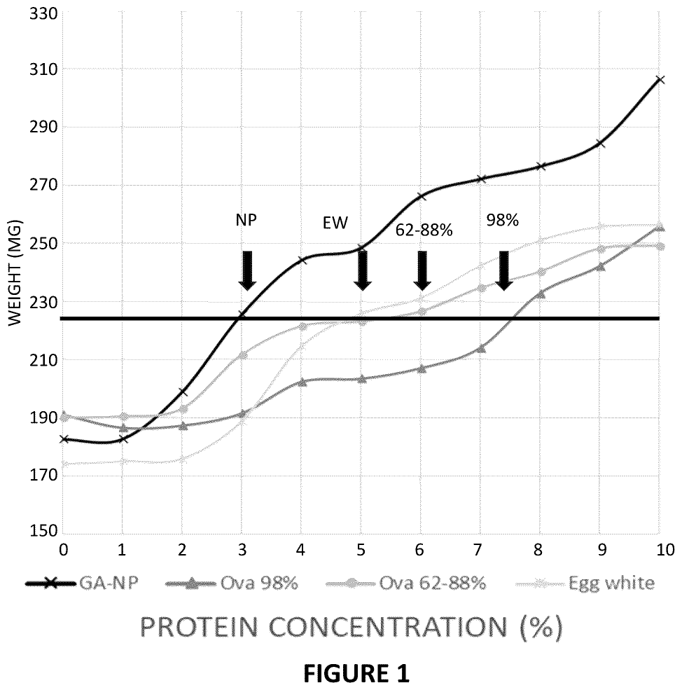

The gelation ability of NP-Ovalbumin produced in yeast was compared to Gallus gallus (GG; chicken) Ovalbumin in three degrees of purity: whole egg white, 62-88% purity, and 98% purity. All proteins were rehydrated to 1-10% (w/w) concentrations. Four hundred (400) μl of each protein were poured into silicon molds and heated for 8.5 minutes at 95° C. in a water bath. Water retention ability was measured by the difference in the weight of each sample before and after heating, and the net weight of the sample was calculated. The results presented in FIG. 1, showing that the NP-Ovalbumin had the best gelation ability from 2% protein in the sample up to 10%, and it had the highest weight. The first arrow from the left indicates that to maintain 225 milligrams of sample only 3% NP ovalbumin solution were required in comparison to 5% of commercial egg white and 6% of 62-88% purity degree of chicken ovalbumin. To this end, 50% less NP-Ovalbumin are required to obtain the same gelation degree in comparison to 62-88% Chicken-ovalbumin and 40% less than Chicken Egg white.

### Example 2

### NP-Ovalbumin has an Irreversible Gelation Ability

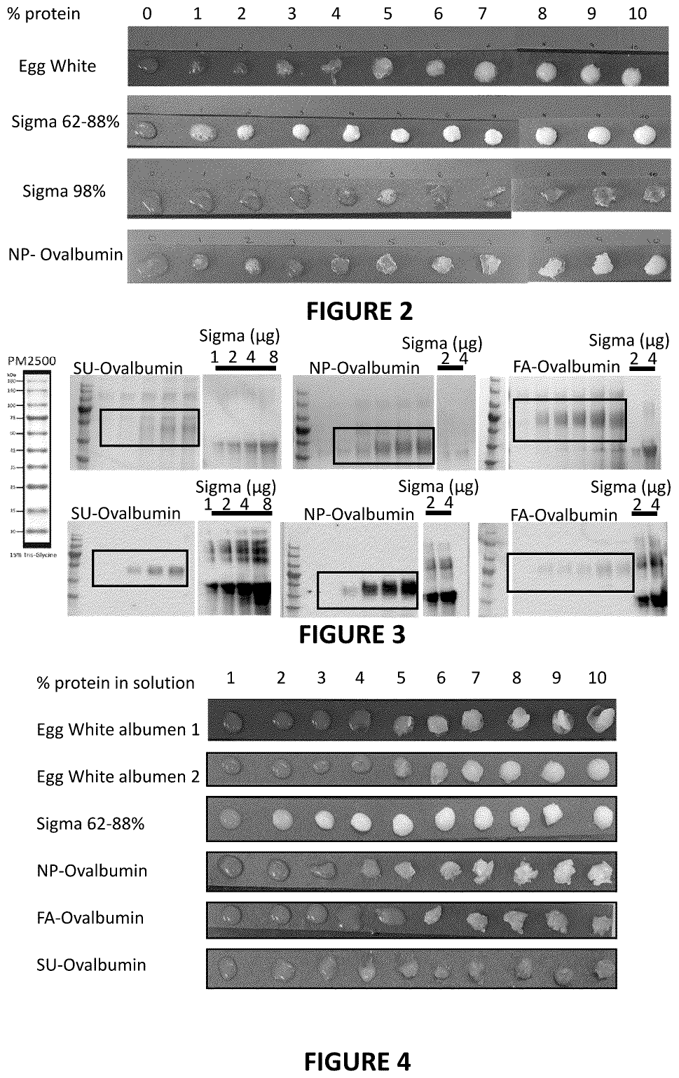

The proteins NP-Ovalbumin produced in yeast together with GG-Ovalbumin (chicken-Ovalbumin) at three degrees of purity: whole Egg white, 62-88% purity, and 98% purity at concentrations of 1-10% w/w were all heated at 95° C. for 8.5 minutes and the ability of the gels to maintain the structure of the mold that it was heated in, was examined. NP-Ovalbumin maintained the structure of the mold at lower concentrations, indicating a better gelation stability (FIG. 2).

After heating all ovalbumins at 95° C. for 8.5 minutes the gel that was formed was stored for up to two months at 4° C. together with Chicken Egg White. In contrast to the Chicken Egg white, NP-Ovalbumin remained in a rigid stable gel form and did not lose liquids that went out of the Chicken egg white gel.

### Example 3

### Fermentation of NP-Ovalbumin Produces Higher Yields Compared to Other Ovalbumins

The inventors have cultivated yeast colonies expressing NP, SU, and FA recombinant ovalbumins, and compared the proteins yield collected from the fermentation medium of each of the different homologues. As shown in FIG. 3 the yield of NP-ovalbumin was much higher than that of both FA and SU-ovalbumin. In the top of FIG. 3, presented are Coomassie blue staining, which marks all the proteins present in the fermentation media. The expression of NP-ovalbumin was to be extremely high, in comparison to the protein standard loaded on the right side of the gel (2 and 4 μg of standard chicken Ovalbumin; FIG. 3). NP-Ovalbumin was also the main protein detected in the fermentation media. Looking at the expression level of both SU and FA-ovalbumin in comparison to the known amount of standard chicken ovalbumin, the yield of both proteins was found to be much lower (FIG. 3). To verify the presence of ovalbumin in the medium, the inventors have also performed a western blot analysis with specific anti-ovalbumin polyclonal antibodies. In the bottom of FIG. 3, the recognition ability of anti-chicken Ovalbumin antibodies to recognize SU and FA-ovalbumin was found to be rather low, and thus, this assay does not fully and/or accurately determines the actual amount of these proteins in the media.

### Example 4

### Comparison Between the Gelation Ability of NP-Ovalbumin and Other Ovalbumins

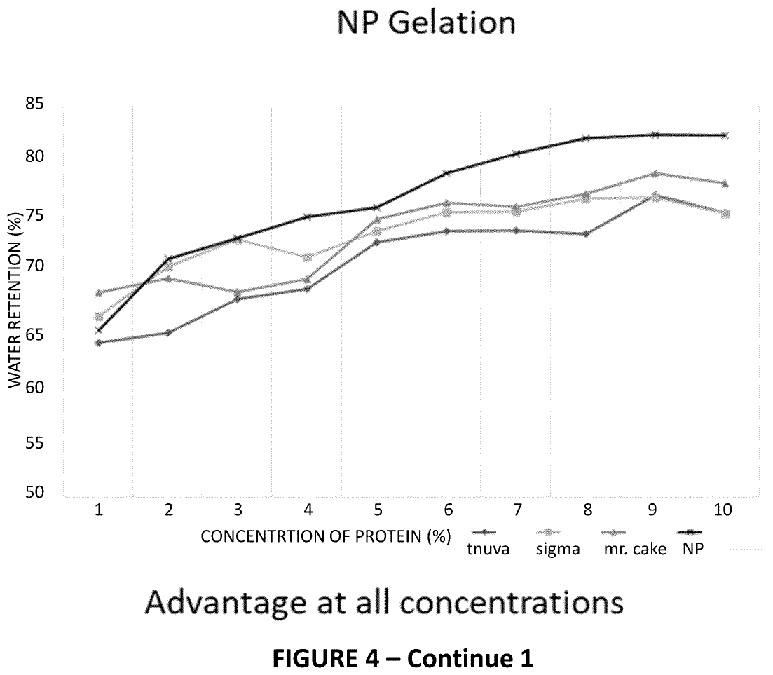

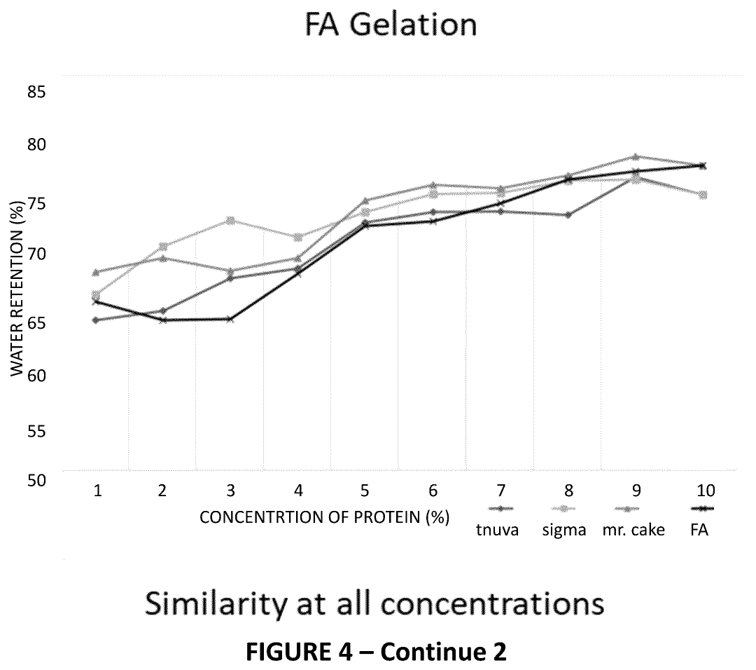

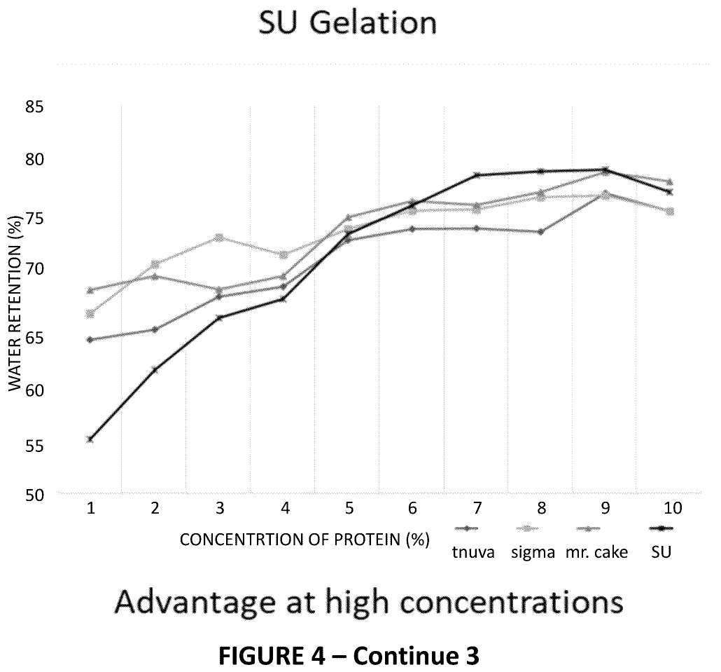

The gelation ability of NP, SU, and FA-Ovalbumins produced in yeast was compared to GG-Ovalbumin (chicken-Ovalbumin) at three degrees of purity: whole Egg white, 62-88% purity, and 98% purity. Four hundred (400) μl of each protein were poured into silicon molds and heated for 8.5 minutes at 95° C. in a water bath. Water retention ability was measured by the difference in the weight of each sample before and after heating and the net weight of the sample was calculated. The results presented in FIG. 4, showing that NP-Ovalbumin had the best gelation ability from 2% protein in the sample up to 10% and it had the highest weight in comparison to GG-Ovalbumin (chicken-Ovalbumin) at three degrees of purity: whole Egg white, 62-88% purity, and 98% purity. SU-Ovalbumin had the best gelation ability from 6% protein in the sample up to 10% and it had the highest weight in comparison to GG-Ovalbumin (chicken-Ovalbumin) at three degrees of purity: whole Egg white, 62-88% purity, and 98% purity. FA-Ovalbumin had similar gelation abilities that resemble GG-Ovalbumin (chicken-Ovalbumin) at three degrees of purity: whole Egg white, 62-88% purity and 98% purity. Summarizing these results, the inventors concluded that NP-Ovalbumin is a better gelation agent than the commercially available chicken Egg white gelling agents, SU-Ovalbumin has an advantage over them at higher concentrations, and FA-Ovalbumin presents similar qualities as the commercially available chicken egg white gelling agents.

### Example 5

### NP-Ovalbumin Increased Gelation Ability Compared to Homologues Ovalbumins

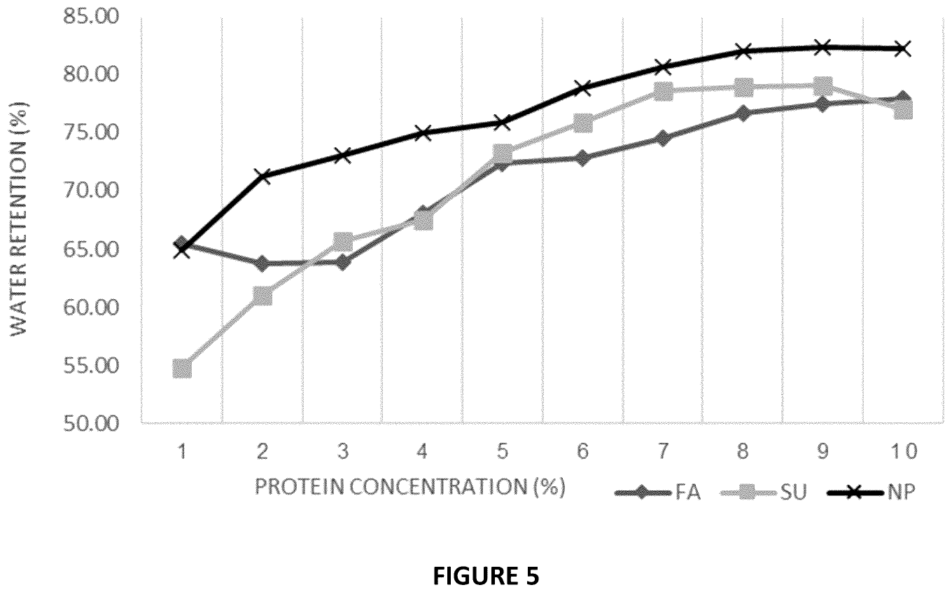

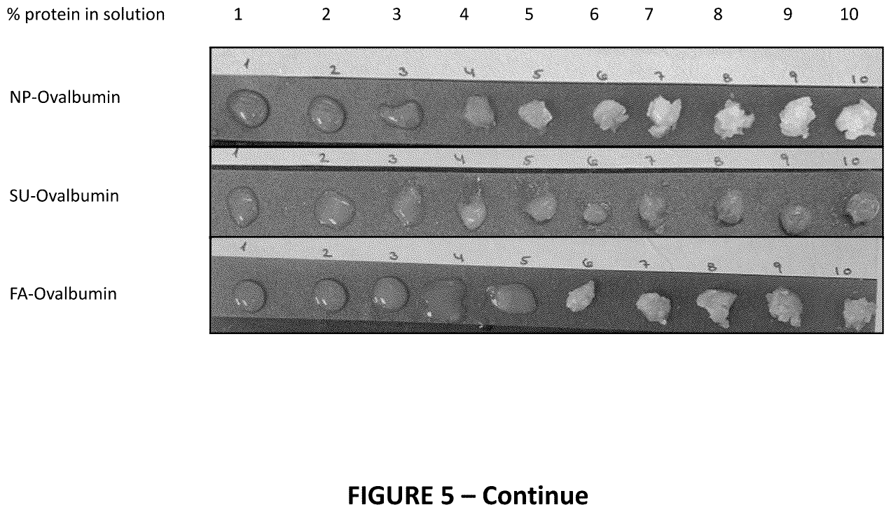

Next the inventors compare the gelation ability of NP, SU and FA-Ovalbumins. Briefly, 400 μl of each protein were poured into silicon molds and heated for 8.5 minutes at 95° C. in a water bath. Water retention ability was measured by the difference in the wight of each sample before and after heating and the net wight of the sample was calculated. The results show that NP-Ovalbumin had the highest gelation ability at all concentrations compared to SU-Ovalbumin and FA-Ovalbumin (FIG. 5). SU-Ovalbumin showed increased gelation ability compared to FA-Ovalbumin at higher concentrations (5-9%, FIG. 5).

### Example 6

### NP-Ovalbumin has Better Water Solubility Compared to Egg White Albumen

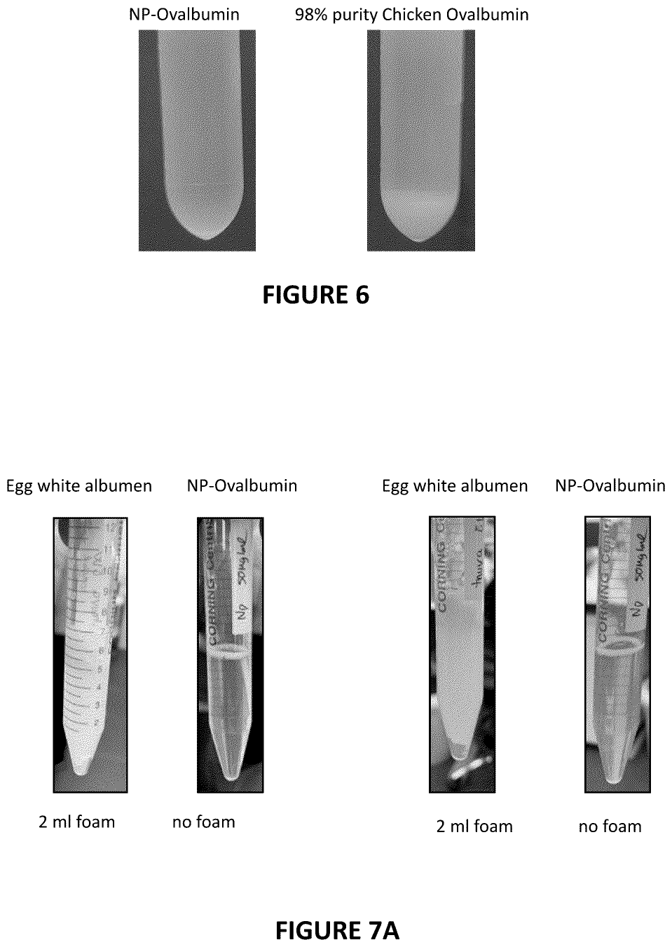

To compare the water solubility of NP-Ovalbumin to Chicken 98% purity ovalbumin, both proteins in a powder form were dissolved in DDW to a final concentration of 10% w/w (100 mg/ml). The solution was homogenized by vortex. After thorough mixing the solutions remained at room temperature for 1 hour. The results show that in contrast to the Chicken 98% purity ovalbumin in which sediments were formed at the bottom of the tube, NP-ovalbumin solution retained clear and homogenous (FIG. 6).

### Example 7

### NP-Ovalbumin has Lower Foaming Ability Compared to Egg White Albumen

In this experiment two egg white proteins were tested: NP-Ovalbumin, as a powder made by fermentation in the lab, and a powder of egg white albumen. Both proteins were dissolved in DDW to a concentration of 5% (50 mg/ml) in a 15 ml test tube until fully homogenized. The total volume of each solution was 5 ml, in which 250 mg of powder were dissolved. The protein solutions were placed untouched for 15 min at room temperature. NP-Ovalbumin solution was transparent, while the egg white albumen solution was opaquer with a yellowish tone. Foaming ability of the protein solutions was measured at 3 different mixing durations: (1) 30 seconds vortex, followed by foam volume measurement; (2) 1 minute vortex, followed by foam volume measurement; and (3) 30 seconds of manual shaking, followed by foam volume measurement.

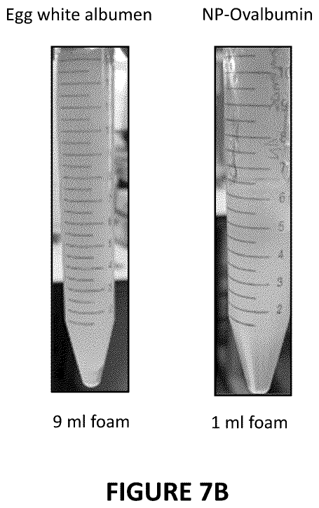

The results in FIG. 7 show that after 30 sec of vortex NP-Ovalbumin solution had barely foamed, while approximately 2 ml of foam were measured in the egg white albumen solution. The same results were observed after one minute of mixing by vortex (FIG. 7A). Following 30 seconds of manual shaking, 1 ml of foam was measured in the NP-Ovalbumin solution (in 15 ml test tube), whereas a greater volume of foam, ˜9 ml, were measured in the egg white albumen solution (FIG. 7B). Overall, NP-Ovalbumin was found to have a substantially lower tendency to foam, compared to egg white albumen solution.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. An edible composition comprising at least one of: recombinant pigeon ovalbumin, recombinant hamerkop ovalbumin, collard flycatcher ovalbumin, or any combination thereof.

2. The edible composition of claim 1, wherein said recombinant pigeon ovalbumin comprises the amino acid sequence set forth in SEQ ID NO: 1, or a functional analog thereof having at least 85% homology thereto.

3. The edible composition of claim 1, wherein recombinant hamerkop ovalbumin comprises the amino acid sequence set forth in SEQ ID NO: 2, or a functional analog thereof having at least 85% homology thereto.

4. The edible composition of claim 1, wherein said recombinant collard flycatcher ovalbumin comprises the amino acid sequence set forth in SEQ ID NO: 3, or a functional analog thereof having at least 85% homology thereto.

5. The edible composition of claim 1, wherein any one of: said pigeon ovalbumin, recombinant hamerkop ovalbumin, and recombinant collard flycatcher ovalbumin is characterized by having a gelation activity equal to or greater than a gelation activity of a control ovalbumin.

6. The edible composition of claim 1, being an egg-white substituent.

7. The edible composition of claim 1, being in the form of a dried powder.

8. The edible composition of claim 1, wherein any one of: said pigeon ovalbumin, recombinant hamerkop ovalbumin, and recombinant collard flycatcher ovalbumin is encoded from a polynucleotide being codon optimized for expression in a cell.

9. The edible composition of claim 8, wherein said cell comprises a yeast cell.

10. The edible composition of claim 1, wherein any one of: said pigeon ovalbumin, recombinant hamerkop ovalbumin, and recombinant collard flycatcher ovalbumin is produced or heterologouslly expressed in a yeast cell.

11. The edible composition of claim 9, wherein said yeast cell comprises a Pichia pastoris cell.

12. A food product or an ingredient thereof comprising the edible composition of claim 1.

13. A method for preparing a food product that requires an amount of egg-white protein, the method comprising substituting an amount of egg-white protein required for preparing said food product with an equivalent amount of the edible composition of claim 1.
